(19) United States
(12) Patent Application Publication
(10) Pub. No.: US 2010/0086596 A1
(43) Pub. Date: Apr. 8, 2010

(54) MICROSPHERES FOR RELEASING AN OCTREOTIDE COMPOUND WITHOUT AN INITIAL TIME LAG

(75) Inventors: Bagavathikanun Chithambara THANOO, Brecksville, OH (US); Gonto JOHNS, III, North Olmstead, OH (US)

Correspondence Address: PEARNE & GORDON LLP, 1801 EAST 9TH STREET, SUITE 1200, CLEVELAND, OH, 44114-3108, US

(73) Assignee: Oakwood Laboratories LLC, Oakwood Village, OH (US)

(21) Appl. No.: 12/245,898

(22) Filed: Oct. 6, 2008

(51) Int. Cl. A61K 9/14; A61K 38/08; A61P 3/00

(52) U.S. Cl. 424/486; 514/16

(57) ABSTRACT

Microspheres for releasing an octreotide compound without an initial time lag include a poly(D,L-lactide-co-glycolide) polymer (PLGA polymer) matrix having a ratio of lactide to glycolide ranging from 80:20 to 90:10 mol %. The polymer has a molecular weight ranging from about 6000 to 16000. The octreotide compound is dispersed in the polymer matrix. The microspheres can be made by forming a dispersed phase by combining the above polymer, dichloromethane, the octreotide compound, methanol and acetic acid. A target loading of the octreotide compound in the dispersed phase ranges from 7 to 12% by weight. Polyvinyl alcohol is dissolved in water to form a continuous phase. The dispersed phase is mixed in the continuous phase to form a microsphere suspension. The dichloromethane, acetic acid, methanol and polyvinyl alcohol are removed from the microsphere suspension. Residual dichloromethane and methanol are removed from the microspheres by washing.

### TECHNICAL FIELD

This disclosure is directed to polymer delivery of active agents, in particular, delivery of octreotide from polymer microspheres without an initial time lag.

### TECHNICAL BACKGROUND

Octreotide is used to treat the symptoms associated with metastatic carcinoid and vasoactive intestinal peptide tumors (VIP-secreting tumors) (Established Clinical Use of Octreotide and Lanreotide in Oncology,” Chemotherapy (2001), 47 (Suppl): 40-53”). Octreotide normalizes the growth hormone levels in acromegaly patients (“Effects of Octreotide Treatment on the Proliferation and Apoptotic Index of GH-Secreting Pituitary Adenomas,” The Journal of Clinical Endocrinology & Metabolism, 86(11): 5194-5200 and “Octreotide Long Acting Release: A Review of its Use in the Management of Acromegaly,” Drugs (2003), 63(22), 2473-2499). Octreotide is indicated for long term maintenance therapy in acromegalic patients for whom medical treatment is appropriate. The goal of treatment in acromegaly is to reduce GH and IGF levels to normal. Octreotide can be used in patients who have had an inadequate response to surgery or in those for whom surgical resection is not an option. It may also be used in patients who have received radiation and have had an inadequate therapeutic response. Octreotide therapy is used in the treatment for diabetic retinopathy (Grant M B, Mames R N, Fitzgerald C, et. al., “The Efficacy of Octreotide in the Therapy of Severe Nonproliferative and Early Proliferative Diabetic Retinopathy,” Diabetes Care, 2000, 23: 504-509). The study showed that octreotide is effective in treating children having hypothalamic obesity by reducing excessive insulin secretion (Lusting R H, Rose S R, Burghen G A, et. al., “Hypothalamic Obesity Caused by Cranial Insult in Children: Altered Glucose and Insulin Dynamics and Reversal by Somastostatin Agonist,” J. Pediatr. 1999; 135: 162-168).

Octreotide is a long acting cyclic octapeptide with pharmacologic properties mimicking those of the natural hormone somatostatin. Octreotide is known chemically as L-cysteinamide, D-phenylalanyl-L-cysteinyl-L-phenylalanyl-D-tryptophyl-L-lysyl-L-threonyl-N-[2-hydroxy-1-(hydroxymethyl)propyl]-, cyclic (2→7)-disulfide; [R-(R*,R*)].

A sustained release octreotide formulation is available commercially in the name of Sandostatin LAR. This formulation improves patients comfort; a single monthly injection is used instead of thrice daily subcutaneous (sc) injection.

Sandostatin LAR uses a custom polymer, a glucose-PLGA “star” polymer that is specially synthesized. Sandostatin LAR does not release the drug for the first two weeks after injection. This requires daily injection to cover the lag time for release. If it is decided to terminate the study due to side effects or other problems, the administered dose takes approximately 70 days or more to clear from the system. The commercially available formulation has only 5% drug content in the microspheres. Approximately 600 mg of microspheres are injected for a 30 mg dose and the injection volume is greater than 2 mL. This might cause excessive pain at the injection site. Additionally, the product requires a large 19G needle for injection into the patient, which might be painful. It would be desirable to develop an improved product that does not have an initial time lag like the commercially available product and does not require using a custom glucose-PLGA star polymer.

### SUMMARY

One embodiment of this disclosure features microspheres for releasing an octreotide compound without a time lag. A poly(D,L-lactide-co-glycolide) polymer (PLGA polymer) matrix has a ratio of lactide to glycolide ranging from 80:20 to 90:10 mol %. The polymer has a molecular weight ranging from about 6000 to 16000. The octreotide compound is dispersed in the polymer matrix. The microspheres are suitable for delivering octreotide compounds for all of their indications and uses.

The words, microsphere, microparticle and microcapsule can be used interchangeably with regard to the invention, and mean encapsulation of the octreotide compound by the polymer; the octreotide compound is dispersed in a matrix of the PLGA polymer. In particular, the term microsphere is used throughout this disclosure.

The term octreotide includes its analogues or derivatives thereof. The terms derivatives and analogues mean branched, straight chain or cyclic polypeptides in which at least one of the amino acids has been omitted or substituted by at least one other amino acid radical(s); and also include at least one functional group being substituted for at least one other functional group(s); and at least one group being substituted by at least one other isosteric group(s). In a broad sense, the terms mean all modified derivatives of octreotide that are biologically active and have a similar effect as unmodified octreotide.

The term “octreotide compound” means octreotide as a free base, salt or complex. Acid addition salts may be formed by inorganic or organic acids or polymeric acids. This includes simple salts (e.g., octreotide acetate, octreotide lactate and octreotide maleate), less soluble salts (e.g., octreotide pamoate) and fatty acid salts (e.g., octreotide palmitate and octreotide stearate). Complexes might be formed by addition of octreotide and inorganic compounds.

Referring to more specific aspects of the first embodiment, the polymer can have an acid end group. In particular, the ratio of lactide to glycolide in the polymer is 85:15 (i.e., PLGA 85:15). The molecular weight of the polymer can be tailored to the particular duration of the formulation. The microspheres are adapted for a 15 day formulation thereof that can employ PLGA polymer having a molecular weight of about 6000-8000. All molecular weight referred to in this disclosure is weight average molecular weight as described herein. The microspheres are adapted for a one month formulation thereof that can employ PLGA polymer having a molecular weight of about 8000 to 14000. The microspheres are adapted for a two month formulation thereof that can employ PLGA polymer having a molecular weight of about 13000 to 16000. In particular, the octreotide compound is octreotide acetate. The microspheres can have an average size ranging from about 25 to 35 microns.

The microspheres that employ PLGA 85:15 polymer are adapted to release octreotide acetate in serum of a rat to a concentration of >1 nanogram per milliliter (ng/mL) in a first day of the release at a dosage of 5 mg per rat. With regard to the one month formulation, the PLGA 85:15 polymer microspheres are adapted to release the octreotide acetate in serum of a rat to a concentration of >3 ng/mL in a first day of the release at a dosage of 5 mg per rat and, in particular, can release the octreotide acetate in serum of a rat to a concentration of >2 ng/mL at the dosage throughout a 30 day release period.

In a specific aspect of the microspheres, the polymer has a ratio of lactide to glycolide of 85:15 mol %. The polymer has a molecular weight ranging from about 6000 to 16000 and an acid end group. Octreotide acetate is dispersed in the polymer matrix.

Another aspect of this disclosure features a method of administering an injectable octreotide compound to a warm blooded species in need thereof (e.g., a mammal including a human) without a time lag. The microspheres described above are provided. Diluent is added as a liquid to the lyophilized microspheres to form a first reconstituted formulation. Alternatively, the microspheres are provided along with diluent in a lyophilized formulation. The lyophilized formulation can be reconstituted with water to form a second reconstituted formulation. Either the first or second reconstituted formulation is injected to the mammal through a needle having an inner diameter of 394 microns or less.

In the product, diluent components comprising sodium carboxymethylcellulose, mannitol and polysorbate are present along with the microspheres. The suspension is advantageously filled and freeze dried with all of the components of the formulation in multiple vials, wherein each vial can include the entire formulation with all components as a single dosage, unlike the conventional injectable octreotide formulation which employs more than one vial of components. This is reconstituted and then injected into the mammal through a needle having an inner diameter≦394 microns corresponding to an inner diameter of a 22 gauge needle (e.g., 22 gauge or a smaller 23 gauge needle). The dimensions of a 19 gauge needle (larger than what is described) are an outer diameter (OD) of 1067 microns and an inner diameter (ID) of 686 microns; a 22 gauge needle has an OD of 711 microns and an ID of 394 microns; and a 23 gauge needle has an OD of 635 microns and an ID of 318 microns.

Another embodiment of this disclosure features a process for preparing microspheres for extended release of an octreotide compound without an initial time lag. Provided is a PLGA polymer having a ratio of lactide to glycolide ranging from 80:20 to 90:10 mol % and a molecular weight ranging from about 6000 to 16000. The dispersed phase is prepared in general by combining the polymer, the octreotide compound, dichloromethane, methanol and acetic acid. However, the dispersed phase solution could be prepared faster by preparing individual solutions of polymer and drug and combining them. In this case, the polymer is dissolved in dichloromethane to form a polymer solution. An octreotide compound is dissolved in a mixture of acetic acid and methanol to form an octreotide solution. The octreotide and polymer solutions are mixed to form a dispersed phase. A target loading of the octreotide compound ranges from 7 to 12% by weight and in particular, 9 to 11% by weight, in the dispersed phase. Target loading is obtained by calculating the amount of drug/the amount of drug and polymer in the dispersed phase (% by weight). Polyvinyl alcohol is dissolved in water to form a continuous phase. For microsphere preparation, the dispersed phase and continuous phase are combined under the influence of mixing, forming microspheres. The dichloromethane, acetic acid and methanol are removed from the dispersed phase droplets immediately under mixing to form a microsphere suspension. Residual solvent (dichloromethane and methanol) in the microspheres is removed by washing with ambient temperature water and hot water (30-40° C.) with or without an air sweep. During washing, polyvinyl alcohol from the continuous phase and solvents released from the dispersed phase to the continuous phase are removed. Microspheres could be isolated by filtration to obtain bulk microspheres for evaluation purposes. Finished product vials could be obtained by suspending the washed microspheres in diluent, adjusting for concentration, filling into vials and freeze drying.

The octreotide microspheres of this disclosure provide many advantages. They are formed using PLGA polymer, not the custom PLGA-glucose star polymer of the prior art. The PLGA polymer used in this disclosure has been carefully studied to select the desired molar ratio of lactide to glycolide and molecular weight that contribute to no initial time lag for release as well as release for the intended duration. Therefore, the daily injections used by conventional octreotide formulations are no longer needed. Another advantage is that various formulations can be prepared (15 day, one month and two month). The 15 day formulation enables faster drug clearing if the patient exhibits an undesirable reaction. Since the entire drug formulation can be filled into a single vial, it can more easily be reconstituted for use. The inventive microspheres also provide the benefit of being injectable using a smaller needle having a size of 22 gauge or less (e.g., 23 gauge), which may avoid pain in patients. While injecting a one month release formulation of PLGA85:15 microspheres in rats at a dose of 5 mg octreotide acetate (per rat), drug level in serum reached more than 3 ng/mL within the first day and the level remained at more than 2 ng/mL for about a one month period. However, Sandostatin LAR took more than 8 days to achieve more than 3 ng/mL concentration for the same dose.

Many additional features, advantages and a fuller understanding of the invention will be had from the accompanying drawings and the detailed description that follows. It should be understood that the above Summary describes the invention in broad terms while the following Detailed Description describes the invention more narrowly and presents specific embodiments that should not be construed as necessary limitations of the invention as broadly defined in the claims.

### BRIEF DESCRIPTION OF THE DRAWINGS

FIGS. 1 and 2 show the results of in vivo studies in rats upon injecting microspheres made using PLGA 50:50 polymer;

FIGS. 3a and 3b compare the results of in vivo studies in rats and in vitro studies in PBS at 37° C., respectively, upon injecting or using microspheres made from PLGA 50:50 polymer:

FIG. 4 shows the results of in vitro studies in PBS at 37° C. for microspheres made using PLGA 50:50 polymer;

FIG. 5 shows the results of in vitro studies in PBS at 37° C. for microspheres made using two PLGA 75:25 polymers;

FIG. 6 shows the results of in vivo studies in rats upon injecting microspheres made using PLGA 75:25 polymer;

FIGS. 7 and 8 show the results of in vivo studies in rats and in vitro studies in PBS at 37° C. upon injecting or using microspheres made from PLA polymer;

FIG. 9 shows the results of in vitro studies in PBS at 37° C. for microspheres made using PLGA 85:15 polymers of the same molecular weight and at different drug loads;

FIG. 10 shows the results of in vivo studies in rats upon injecting microspheres made using PLGA 85:15 polymer at the relatively high and low molecular weights of 13,900 and 7,900;

FIG. 11 shows the results of in vivo studies in rats upon injecting microspheres made using PLGA 85:15 polymers from multiple lots, one sample with dimethylsulfoxide (DMSO) in the dispersed phase and also the Sandostatin LAR formulation; and

FIG. 12 shows the results of in vivo studies in rats upon injecting microspheres made using PLGA 85:15 polymer on a pilot scale.

### DETAILED DESCRIPTION

This disclosure features microspheres for releasing an octreotide compound (e.g., octreotide acetate) either in vitro or in vivo without an initial time lag. The microspheres include PLGA polymer in which a ratio of lactide to glycolide ranges from 80:20 to 90:10 mol %. The PLGA polymer has a molecular weight ranging from 6000 to 16000 daltons. Another feature of the polymer is that it has an acid end group that is not blocked. A specific polymer especially suitable in this disclosure is a PLGA 85:15 polymer. The octreotide compound is dispersed in the polymer matrix of the microspheres. The molecular weight of the polymer is the weight average molecular weight determined by GPC using polystyrene standards and Tetrahydrofuran (THF) as the solvent.

The PLGA polymer (80:20 to 90:10) used in the microspheres, which is biodegradable and biocompatible, can be tailored to the duration of release of the formulation. A 15 day formulation of the microspheres employs PLGA polymer having a molecular weight of 6000-8000 daltons. A one month formulation of the microspheres employs PLGA polymer having a molecular weight of 8000 to 14000 daltons. A two month formulation of the microspheres employs PLGA polymer having a molecular weight of 13000 to 16000 daltons. Even though the release duration is up to one month for PLGA 85:15 having a molecular weight of 8000, this formulation could be used for a 15 day repeat injection since the drug level starts declining after 15 days. Similarly, PLGA 85:15 having a molecular weight of 14000 or more could be used for two month release formulation.

This disclosure achieves sustained release octreotide microspheres with improved performance using a standard type of, but specially selected, PLGA polymer, which is available from multiple manufacturers (e.g., Boehringer Ingelheim, Lakeshore Polymers, Purac and Alkermes).

Another feature is that a target loading of drug in the dispersed phase ranges from 7 to 12% by weight, more specifically, from 9 to 11% by weight. The target load of 8.5 to 12% produced microspheres with appropriate initial release. The microspheres have an average size ranging from about 25 to 35 microns.

Another embodiment of this disclosure is an injectable formulation and method that include the octreotide microspheres. The entire formulation that is filled into a single vial, upon reconstitution, is adapted to be injected into a mammal through a needle having a size of 22 gauge or less (394 micron inner diameter or less). This advantageously should avoid pain when administering octreotide to a patient.

The “lyophilized pharmaceutical formulation” according to the disclosure can be administered intramuscularly, subcutaneously, or orally in the form of a suspension in a suitable liquid carrier. Accordingly, also provided by the disclosure is a method of treating a disease, disorder or condition in a warm blooded species (e.g., a mammal including a human patient) in need of such treatment. This method comprises use of the pharmaceutical formulation of the disclosure to administer an octreotide compound to the patient. While any suitable means of administration to a patient can be used within the context of the disclosure, typically the inventive method of treating a disease in a patient involves administering the pharmaceutical formulation to a patient via injection. By the term “injection,” it is meant that the composition is forcefully introduced into a target tissue of the patient. The composition can be administered to the patient by any suitable route, but is specifically administered to the patient intramuscularly or subcutaneously. When the inventive pharmaceutical formulation is administered by injecting, any suitable injection device can be used. Other routes of administration can be used to deliver the composition to the patient in accordance with the inventive method. Indeed, although more than one route can be used to administer the inventive formulation, a particular route can provide a more immediate and more effective reaction than another route.

According to yet another aspect of the disclosure, a pharmaceutical formulation and a method of producing it are provided. The pharmaceutical formulation utilizes a container, e.g., containing a single dose of microspheres containing an octreotide compound for treating a condition that is treatable by the sustained release of octreotide active agent from the microspheres and suspending agents. The amount of microspheres and suspending agents in the single dose is dependent upon the amount of active agent present in each container. Specifically, the single dose is selected to achieve the sustained release of the active agent over a period of from 15 days, one month or 2 months with the desired release profile.

The microspheres can be administered alone, or in appropriate combination with other active agents or drug therapies, as part of a pharmaceutical formulation. Such a pharmaceutical formulation may include the microspheres in combination with any standard physiologically and/or pharmaceutically acceptable carriers which are known in the art. The formulation compositions preferably are sterile and contain a therapeutically effective amount of the microsphere in a unit of weight or volume suitable for administration to a patient. The term “pharmaceutically-acceptable carrier” as used herein means one or more compatible solid or liquid fillers, diluents or encapsulating substances which are suitable for administration into a human or other mammal. The term “carrier” denotes an organic or inorganic ingredient, natural or synthetic, with which the active ingredient-containing microspheres are combined to facilitate the application. The components of the pharmaceutical formulation preferably are capable of being co-mingled with the components of the present disclosure (e.g., the active agent, the biodegradable polymer), and with each other, in a manner such that there is no interaction that substantially impairs the desired pharmaceutical efficacy. Pharmaceutically acceptable carrier further means a non-toxic material that is compatible with a biological system such as a cell, cell culture, tissue, or organism. The characteristics of the carrier depend on the route of administration. Physiologically and pharmaceutically acceptable carriers include diluents, fillers, salts, buffers, stabilizers, desiccants, bulking agents, propellants, acidifying agents, coating agents, solubilizers, and other materials which are well known in the art. Carrier formulations suitable for oral, subcutaneous, intravenous, intramuscular, or other type of administrations also are well known, and can be found, e.g., in Remington's Pharmaceutical Sciences (Mack Publishing Co., Easton, Pa.), as well as in other sources.

The “pharmaceutically-acceptable carrier” according to the disclosure can be bulking agents and wetting agents, for example, sodium carboxymethylcellulose and mannitol. In a suspension including the octreotide microspheres, diluent and water, mannitol is present in an amount, for example, of 40 to 80 mg/mL (4 to 8%), more specifically, 45 to 65 mg/mL (4.5 to 6.5%) of the suspension; sodium carboxymethylcellulose can be present in an amount, for example, of 5 mg/mL (0.5%), more specifically, 3 to 10 mg/mL (0.3 to 1%) of the suspension; and polysorbate can be present in an amount, for example, of 0.05 to 0.1% of the suspension.

In a finished lyophilized product vial a lyophilized formulation can include, for example, sodium carboxymethylcellulose in an amount of 0.1% to 10%, even more specifically about 1.5% to about 5.0% by weight of the formulation, mannitol can be present in an amount, for example, of 10% to 50%, even more specifically about 18% to about 21% by weight of the formulation. More specifically, the lyophilized formulation can contain 70% (280 mg) of octreotide microspheres, 25% mannitol (100 mg), 2.5% (10 mg) sodium carboxymethylcellulose and 0.2% between 80 (1 mg) by weight of the formulation.

Preparations for parenteral administration include but are not limited to sterile aqueous or non-aqueous solutions, suspensions, and emulsions. Examples of solvents include propylene glycol, polyethylene glycol, and vegetable oils such as olive oil, injectable organic esters such as ethyl oleate, and the like. Aqueous carriers include water, salts and buffer solutions such as saline and buffered media, alcoholic/aqueous solutions and emulsions or suspensions, as well as others. Parenteral vehicles include but are not limited to Normal Saline (0.9% sodium chloride), ½ Normal Saline (0.45% sodium chloride), 5% Dextrose in Water, Lactated Ringer's Solution, 5% Dextrose in ½ Normal Saline with 20 mEq KCl, 5% Dextrose in Lactated Ringer's Solution, 5% Dextrose in ⅓ Normal Saline, 5% dextrose in ½ Normal Saline, Normosol®-M in 5% Dextrose, Normosol®-R in 5% Dextrose, as well as others. Intravenous vehicles include fluid and nutrient replenishers, electrolyte replenishers (such as those based on Ringer's dextrose), and the like. Preservatives and other additives also optionally can be present such as, for example, antimicrobials, anti-oxidants, chelating agents, and inert gases and the like, so long as these additional ingredients do not deleteriously impact the advantageous properties of the microspheres. The “reconstitution solvent” according to the disclosure can be an aqueous carrier, preferably water for injection. The amount of water for injection can be used for reconstitution and ranges from about 1 mL to about 5 mL, even more specifically about 1 mL to about 2 mL.

The “octreotide loaded microspheres” according to the disclosure generally have a spherical shape and range in size from about 0.1 microns to about 500 micrometers in diameter, even more specifically from about 1 to about 200 microns, depending upon the fabrication conditions. The octreotide content in the octreotide loaded microspheres ranges from 7% to 10% of weight of the microspheres. The microspheres can be employed as a “delivery system” to release active agent from the interior of the microsphere (it can be released from the interior and exterior of the microspheres, e.g., a surface associated drug), when placed in an appropriate aqueous medium (e.g., such as in body fluids, in a physiologically acceptable buffer, or in any appropriate aqueous environment). As used herein, the term “sustained-release” refers to the release of an active agent from the microspheres of the disclosure over a defined or extended period of time in a continuous, discontinuous, linear or nonlinear manner. Methods of measuring release are well known in the art (see, e.g., Hora et al., Pharm. Res. 7:1190-1194 (1990); Hora et al., Bio/Technology 8:755-758 (1990)). According to the disclosure sustained release can be continuous, relatively linear, and prolonged (i.e., as opposed to being short-lived).

The microspheres are made as follows. A dispersed phase is made by dissolving polymer and octreotide acetate in a solvent mixture. The PLGA polymer is dissolved in a suitable solvent (e.g., dichloromethane, methylene chloride, chloroform, ethyl acetate, substituted pyrrolidone, N methyl pyrolidone, polyethylene glycol, acetonitrile, acetone, ethyl methyl ketone, DMSO, dimethyl formamide or dimethyl acetamide). The octreotide acetate or somatastatin analog (e.g., lanreotide or vapreotide) is dissolved in acid (e.g., acetic acid) and a suitable solvent (e.g., methanol, ethanol, DMSO, dimethyl formamide or dimethyl acetamide). In general, the solvent for the drug is a nonsolvent for the polymer and the solvent for the polymer is a nonsolvent for the drug. However, some solvents such as DMSO, DMF, DMAc or acetonitrile could be a solvent for both polymer and drug. The polymer and octreotide solutions are true, filterable solutions. It will be apparent that the octreotide compound, polymer, their solvents and the acid could be added separately or all together at the same time. The selection of particular solvents and continuous phases can be varied depending upon the intended product characteristics.

Small scale batches (<5 g scale) are prepared as a batch process using a standard homogenizer mixer. In the process, the continuous phase is charged in a vessel and a standard Silverson homogenizer (Model L4RT from Silverson Machines) equipped with a standard mixing head and emulsor screen are immersed into the continuous phase. While mixing, the dispersed phase is introduced just below the mixing head using a long needle producing microspheres. Residual solvent and PVA are removed by optional air sweep and washing (ambient at elevated temperature) (for batch processing). Large scale batches (5 g and higher) use a specially designed in-line Silverson mixer (for continuous processing) as disclosed in U.S. Pat. No. 5,945,126, which is incorporated herein by reference in its entirety. To the specially designed Silverson homogenizer continuous phase and dispersed phase are introduced simultaneously while mixing. Microspheres are formed in the in-line Silverson mixer. The continuous process adds to the modified Silverson homogenizer the dispersed phase and the continuous phase at certain flow rates specified in the U.S. Pat. No. 5,945,126. The U.S. Pat. No. 5,945,126 may be referred to for various aspects of the disclosed continuous process of making microspheres.

The dispersed phase is mixed with the continuous phase to form a microsphere suspension. The suspension is believed to be formed by nearly instantaneous emulsification of the dispersed phase in the continuous phase. The dispersed phase is dispersed or emulsified in the continuous phase to form droplets or inclusions of the dispersed phase in the continuous phase. The terms emulsified or dispersed are intended in their broadest sense as meaning discrete regions of dispersed phase interspersed within the continuous phase. The noted inclusions will occur as generally spherical droplets, but in some instances may be irregular inclusions due to particular emulsification conditions. Any suitable medium in which the dispersed phase will form droplets or inclusions may be used as a continuous phase, with those that provide a maximum solvent sink for the dispersed phase solvent being especially desirable. Hence, the flow rate ratio of continuous phase to that of dichloromethane (CP/DCM Ratio) in the dispersed phase is ≧50 since the solubility of dichloromethane in water is around 2%. The continuous phase might also contain surfactant, stabilizers, salts or other additives that modify or affect the emulsification process.

The particular continuous phase is primarily water. The aqueous continuous phase will typically contain a dissolved stabilizer, such as polyvinyl alcohol in an amount of from about 0.1% to about 5%. The continuous phase could contain other surfactants such as polysorbates (Tween), sodium oleate or disodium octylsulfosuccinate.

After the dispersed phase addition is complete, the microsphere suspension is mixed at a lower speed for solvent removal. This could be carried out in a solvent removal vessel (e.g., an Applikon bioreactor). Solvent removal is achieved by exchanging the continuous phase with room temperature water, followed by hot water (30-40° C.), followed by room temperature water. The room temperature water removes external phase solvent; the hot water removes internal residual solvent in the microspheres and then the microspheres are returned to room temperature water for further processing. An optional air sweep is used at the surface of the stirring suspension to remove the headspace solvent during the solvent removal process. Efficient solvent removal could also be achieved by washing alone without an air sweep for the headspace. The microspheres are filtered on a Durapore membrane filter using an Amicon stir cell assembly. The microspheres are washed with water to remove residual stabilizer (e.g., PVA). They are then dried at low temperature (<25° C.) under a vacuum.

The solidified microspheres containing octreotide are uniformly suspended in a diluent solution that contains sodium carboxymethylcellulose and mannitol. The concentration of mannitol in the microsphere suspension ranges from about 10 mg/g to 100 mg/g, preferably 30 mg/g to 60 mg/g. The concentration of sodium carboxymethylcellulose in the microsphere suspension ranges from about 1 mg/g to 20 mg/g, preferably 2 mg/g to 15 mg/g. The suspension of octreotide-loaded microspheres are filled into a container, e.g. glass vials, and lyophilized.

The lyophilized formulation of the present disclosure is a white to slightly yellow lyophilized cake or powder of octreotide containing PLGA microspheres, sodium carboxymethyl cellulose and mannitol. The lyophilized octreotide of the present disclosure preferably has a purity of about 90% or greater (i.e., contains about 10% or less of total impurities based on the total weight of octreotide), more specifically has a purity of about 95% or greater. Purity is determined by high performance liquid chromatography assay (e.g., allowing separation of pure lyophilized octreotide from impurities, and quantitation of the relative amounts by the determination of the peak area of pure octreotide as compared to total peak area), or by a similar method and excludes moisture in the octreotide acetate, and the acetate itself.

The lyophilized octreotide sustained release microsphere formulation can comprise any suitable amount of octreotide, but ideally comprises a therapeutically effective amount of octreotide. A “therapeutically effective amount” means an amount sufficient to show a meaningful benefit in an individual, e.g., promoting at least one aspect of treatment, healing or prevention of other relevant medical condition(s) such as that associated with acromegaley and cancer syndromes. Therapeutically effective amounts may vary depending upon the biological effect desired in the individual, condition to be treated, and the individual. In this regard, the octreotide in the lyophilized microspheres can be present in the sustained formulation in an amount from about 5 mg to about 50 mg (e.g., about 5 mg, about 10 mg, about 20 mg, about 30 mg, or about 50 mg). More specifically, the lyophilized octreotide is present in an amount from about 10 mg to about 30 mg (e.g., about 10 mg, about 20 mg, or about 30 mg).

The lyophilized octreotide microsphere formulation has low moisture content. The moisture content of the inventive lyophilized octreotide microsphere formulation is the result of residual water that remains in the formulation after the lyophilization process. The moisture content can be the product of any suitable solvent that is used in the method of producing the lyophilized octreotide microsphere formulation described herein. The lyophilized octreotide microsphere formulation can have a moisture content of less than from about 0.01 wt % to about 10 wt %, where the wt % is the % water relative to the dry weight of the lyophilized octreotide microsphere formulation. The moisture content can be less than from about 2 wt %, more specifically, less than 1% wt %.

The inventive lyophilized octreotide microsphere formulation according to the disclosure can be contained within a sealed container. Each formulation can be contained within a container that is sealed aseptically. The container can be provided with an opening and a means for aseptically sealing the opening, e.g., such that the sealed container is fluidly sealed or the sealed opening is substantially impermeable to atmospheric gasses, moisture, pathogenic microorganisms, or the like. The container can be constructed of any suitable material such as, for example, glass, polypropylene, Daikyo Resin CZ (sold by Daikyo Gomu Seiko, Ltd.), polyethylene terephthalate, and the like. In particular, the container is constructed of glass. Suitable glass containers include, but are not limited to, glass vials.

A suitable means for sealing the container can include, for example, a stopper, a cap, a lid, a closure, a covering which fluidly seals the container, or the like. Examples of suitable closures include closures that are suitable for medical vials, such as those described in U.S. Pat. No. 4,671,331, and references cited therein. The means for sealing the container are not limited to separate closures or closure devices, but also includes self-sealing containers and containers which are manufactured and sealed during filling operations. The means for aseptically sealing the container can include a stopper such as, for example, a stopper that is configured to fluidly seal the opening.

An outer seal is provided which covers and entirely surrounds the stopper. The outer seal can be constructed of any suitable material. When an outer seal is used, it is fitted with a lid that can be easily manually removed to provide access to the stopper. Such seals include an outer rim made of a suitable material, such as aluminum, that entirely surrounds the lateral edge of the stopper and further include a lid (typically polypropylene or other suitable material) that entirely covers the upper surface of the stopper. The polypropylene lid can be “flipped” off e.g., by exerting upward pressure with a finger or thumb, to provide access to the stopper, e.g., so that it can be punctured with a hypodermic needle to deliver an aqueous vehicle for constitution (see, e.g., U.S. Pat. No. 6,136,814).

The disclosure further provides a solution prepared by suspending the inventive lyophilized octreotide microsphere formulation in an aqueous vehicle. The aqueous vehicle is preferably a sterile aqueous vehicle that is normally used as liquid vehicle for injection. Suitable aqueous vehicles include, for example, sterile water (e.g., Sterile Water for Injection, USP), sodium chloride solutions (e.g., 0.9% Sodium Chloride for Injection, USP), dextrose solutions (e.g., 10% Dextrose for Injection), sodium chloride/dextrose mixtures (e.g., 5% Dextrose and 0.225% Sodium Chloride for Injection, 5% Dextrose and 0.45% Sodium Chloride for Injection), Lactated Ringer's for Injection, and mixtures thereof

The inventive lyophilized octreotide microsphere formulation can be suspended in any suitable volume of the aqueous vehicle. Specifically, the lyophilized octreotide microspheres are suspended in about 10 mL or less (e.g., about 10 mL, about 8 mL, about 6 mL, about 4 mL, or about 1 mL) of the aqueous vehicle. The lyophilized octreotide microspheres can be suspended in about 1 mL to about 5 mL of the aqueous vehicle. More specifically, the lyophilized octreotide acetate is suspended in about 2 mL to about 3 mL of the aqueous vehicle.

The disclosure will now refer to the following examples, which should not be used to limit the claimed invention.

### EXAMPLE 1

Co-Monomer Ratio, Molecular Weight and End Group

Several microsphere batches were prepared using polymers having lactide content varying from 50% (PLGA 50:50) to 100% (PLA) and where molecular weight varied from 7,000 to 50,000 daltons. Table 1 shows the polymer details. Table 2 shows the preparation parameters. Table 3 shows the drug release properties under in-vitro and in-vivo conditions.

The co-monomer ratio and end group of the polymer employed here are those that were certified by the polymer manufacturer. Weight average molecular weight (Mw) of the polymer was determined by size exclusion chromatography (SEC) which is gel permeation chromatography (GPC). Molecular weight was determined by preparing the polymer solution in tetrahydrofuran (THF). Molecular weight separation was performed using Styragel columns from Waters Inc. and two columns HR-4 and HR-2 were used in series. Narrow molecular weight polystyrene standards were used for calibration. The mobile phase was THF.

TABLE 1
Properties of Polymers Used During Initial Study

| Co- | | | | |
|---|---|---|---|---|
| monomer | | | | |
| (L:G | | | | |
| Ratio) | Polymer Code | Manufacturer | End group | Mw |
| 50:50 | RG502H | Boehringer Ingelheim | Free acid | 11,000 |
| | RG503H | Boehringer Ingelheim | Free acid | 30,000 |
| | RG503 | Boehringer Ingelheim | End- | 32,000 |
| | | | blocked | |
| | RG504H | Boehringer Ingelheim | Free acid | 45,000 |
| | 50:50DL2.5A | Alkermes/Medisorb | Free acid | 22,000 |
| 75:25 | PLGA75:25H | Boehringer Ingelheim | Free acid | 14,000 |
| | 7525DL2.5A | Alkermes/Medisorb | Free acid | 25,000 |
| 85:15 | PLGA85:15 | Birmingham Polymers | Free acid | 17,000 |
| | 85:15DL2A* | Alkermes/Medisorb | Free acid | 14,000 |
| 90:10 | PLGA90:10 | Birmingham Polymers | Free acid | 7,000 |
| 100:0 | 100DL2A | Alkermes/Medisorb | Free acid | 14,000 |
| | PLA | Birmingham Polymers | Free acid | 7,000 |

*Multiple lots having varying Mw ranging from 7900 to 14,000 were used at a later stage

Several microsphere batches were prepared by an Oil-in-Water (O/W) process using the polymers listed in Table 1. This was performed to identify appropriate polymers for octreotide microspheres for one month release. Selected microsphere batches were tested in rats for the release profile. Microsphere batches were also tested in-vitro.

In-vitro release under physiological conditions was performed in phosphate buffered saline (PBS) which also contains a small amount of sodium azide as a preservative and polysorbate-80 as the wetting agent. The release medium was a 0.02M Phosphate buffer, pH 7.4, which also contained 0.003M KCl, 0.14M NaCl, 0.5% Tween-80 and 0.5% sodium azide. After adjusting the pH to 7.4 using NaOH or phosphoric acid, the buffer was sterile filtered.

To perform the release, to 120 mg microspheres in a 20 mL screw capped vial 10 mL release medium was added. The contents were then incubated at 37° C. in a shaking water bath (100-120 rev/min). At each time point sample tubes were removed from the bath and allowed to stand for approximately 10 minutes. Approximately 9 mL supernatant were removed using a glass transfer pipette to another 12 mL tube with special care to avoid microsphere removal from the system. The supernatant was then centrifuged and an aliquot was transferred to HPLC vials for assay. After taking the HPLC sample, the entire supernatant was carefully removed and discarded. Care was taken not to discard particles if any were at the bottom of the tubes.

To the tube 9 mL fresh release media was added and mixed well to suspend the settled particles. This was then transferred to the original 20 mL in vitro release tube, and then the 20 mL tubes are placed back in the shaking water bath. A study was performed with duplicate samples. The % released octreotide was calculated from the octreotide input initially made in the 120 mg sample.

In-vivo release studies were performed in rats by subcutaneous injection. Sprague Dawley rats were injected with octreotide microspheres suspended in diluents (carboxymethyl cellulose, mannitol, and polysorbate-80). The dose was generally 1.5 mg octreotide unless specified. After injection, blood samples were collected from the rats and the blood sample was processed to collect serum. Octreotide level in serum was assayed by Radioimmuno assay (RIA). The drug release profile in rat was evaluated.

Microsphere Preparation

The dispersed phase was prepared by dissolving polymer and octreotide acetate in a solvent mixture. The polymer was dissolved in dichloromethane (DCM) and the octreotide acetate was dissolved in methanol (MeOH). The batch size was 2 g which is the combined weight of polymer and octreotide contained in the dispersed phase (DP). The continuous phase (CP) was prepared by dissolving PVA in water; higher temperature (e.g., 70° C.) was used to achieve dissolution. The compositions of the DP and CP are provided in Table 2. The CP (1.5 L) was charged in a 2-3 L vessel equipped with temperature control. A Silverson homogenizer (Model L4RT from Silverson Machines) equipped with a standard emulsor screen was immersed in the CP. The DP was drawn into a syringe and added to the CP while mixing at the RPM shown in Table 1, just below the mixing head using a long (12″) syringe needle bent appropriately to reach the position below the mixing head. After the DP addition was complete, the microsphere suspension was mixed at a lower speed (e.g., 500 RPM) for solvent removal. Solvent removal was performed by heating the suspension to the temperature of about 40° C. and holding at the temperature for one hour. An air sweep was used at the surface of the stirring suspension. The microspheres were filtered on a Durapore membrane filter using an Amicon stir cell assembly. The microspheres were washed with water to remove residual PVA. They were dried at low temperature (<25° C.) under a vacuum.

TABLE 2
Microsphere Batches Prepared with PLGA 50:50 Polymers

| | | | | | | 50:50 | |
|---|---|---|---|---|---|---|---|
| | | RG502H | | RG503H | RG503 | DL2.5A | RG504H |
| | | A | B | C | D | E | F |
| DP | Polymer | 0.29 | 0.25 | 0.16 | 0.17 | 0.21 | 0.15 |
| Composition | Octreotide | 0.04 | 0.03 | 0.02 | 0.02 | 0.02 | 0.02 |
| g/g | DCM | 0.60 | 0.64 | 0.74 | 0.73 | 0.70 | 0.76 |
| | MeOH | 0.06 | 0.07 | 0.08 | 0.08 | 0.07 | 0.08 |
| DP | MeOH/DCM | 0.1 | 0.1 | 0.1 | 0.1 | 0.1 | 0.1 |
| Parameters | Target Load | 13 | 10 | 11 | 11 | 10 | 11 |
| CP | PVA, g/g | 0.0035 | 0.0035 | 0.0035 | 0.0035 | 0.0035 | 0.0035 |
| Composition | | | | | | | |
| Process | Mixing speed | 5500 | 6000 | 5500 | 5500 | 7000 | 5500 |
| parameters | Solvent | Air | Air | Air | Air | Air | Air |
| | removal | sweep, | Sweep, | Sweep, | sweep, | sweep, | sweep, |
| | | ≈40° C. | ≈40° C. | ≈40° C. | ≈40° C. | ≈40° C. | ≈40° C. |
| Finishing | Recovery | Filtration | Filtration | Filtration | Filtration | Filtration | Filtration |
| Drug content in MS (%) | | 8.6 | 9.0 | 10.1 | 3.3 | 8.3 | 9.2 |
| Drug encap. Efficiency | | 66 | 90 | 91 | 30 | 83 | 84 |
| Particle | 10% under | 6 | 2 | 3 | 5 | — | 2 |
| Size, | 25% under | 12 | 7 | 11 | 11 | — | 6 |
| Volume | 50% under | 24 | 16 | 21 | 21 | — | 17 |
| distribution, | 75% under | 36 | 23 | 31 | 31 | — | 29 |
| Micron | 90% under | 47 | 28 | 41 | 39 | — | 39 |

| | | | | | | |
|---|---|---|---|---|---|---|
| Bulk density | 0.19 | 0.37 | 0.72 | 0.34 | 0.71 | |
| % Impurity | 1.4 | 0.9 | 6.7 | 14.0 | 5.9 | 7.9 |

Drug content in the microsphere (%) is the percentage of octreotide acetate present in the microspheres. Drug content in the microsphere was determined by dissolving the known amount of microspheres (example 20 mg) in DMSO (e.g., 7 g) and extracting the drug into acetate buffer (0.1 M, pH 4) (e.g., 13 g). The cloudy extract was centrifuged or filtered to obtain a clear solution for HPLC. The sample was then assayed by HPLC against a calibration standard of octreotide acetate. From the amount of octreotide acetate in the total extraction medium and the weight of the microspheres, the drug load in the microspheres was calculated.

RG503 Polymer, Batch (Sample D)

Microsphere batches should have acceptable drug encapsulation efficiency; at least 60% encapsulation efficiency is appropriate, preferably 70% or higher. This polymer was unsuitable for the formulation due to poor encapsulation efficiency and high impurities. Almost all the impurities in the octreotide microspheres are octreotide related substances, compounds formed between octreotide and the fragments of PLGA (monomer and oligomers). RG503 is an end blocked polymer, whereas an acid end group polymer is desired.

RG504H and RG503H Polymer, Batches (Samples F and C)

These polymers were subjected to an octreotide release study. Each batch was dosed to 24 rats at 1.5 mg octreotide acetate/rat. Microspheres were suspended in diluents (mannitol, carboxymethyl cellulose and polysorbate-80) for injection. Rats were injected with pentobarbital 30 minutes prior to blood sampling. For each blood sampling point six rats were used. Rats were dedicated for sample points. Assay for octreotide in rat blood was performed by RIA.

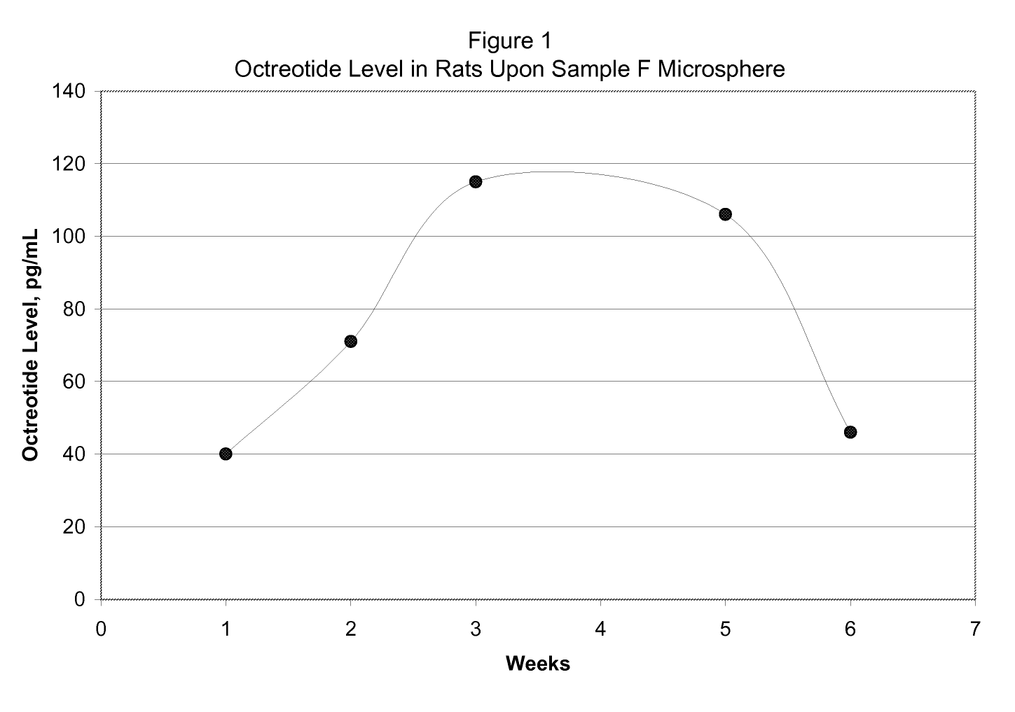

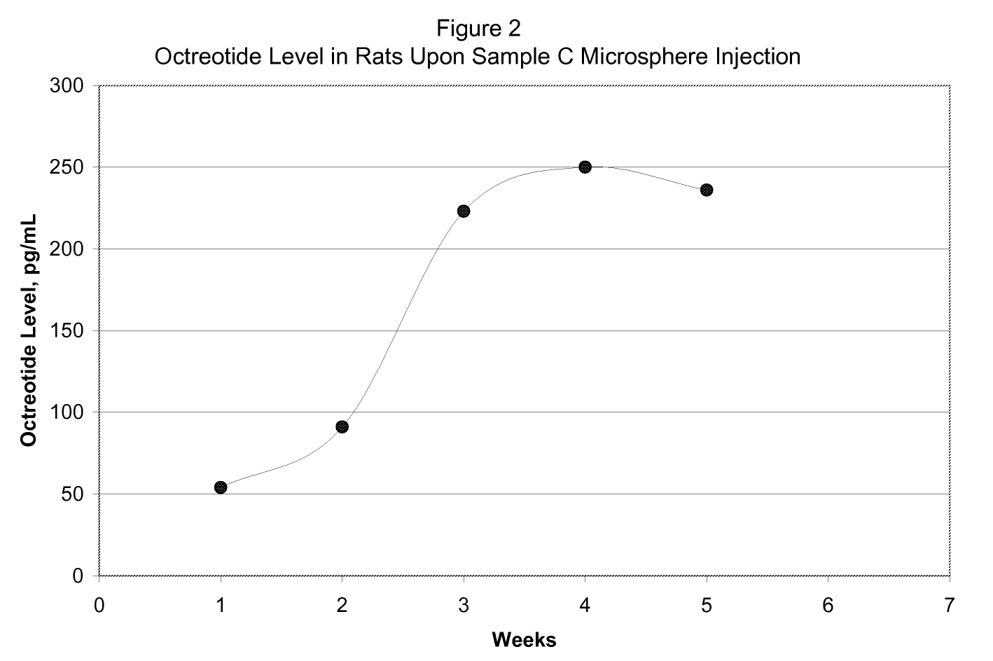

FIG. 1 compares the in-vivo release profile in rats for Sample F (RG504H batch). Similarly, FIG. 2 compares in-vivo release profile in rats for Sample C, batch produced from RG503H. The results show that the batches had a lag time for release. Duration of the release appeared to be 4-6 weeks. Thus, RG503H and RG504H polymer based microspheres had a lag time for release and the release duration extended to greater than 6 weeks.

RG502H Polymer, Batch (Sample B and A)

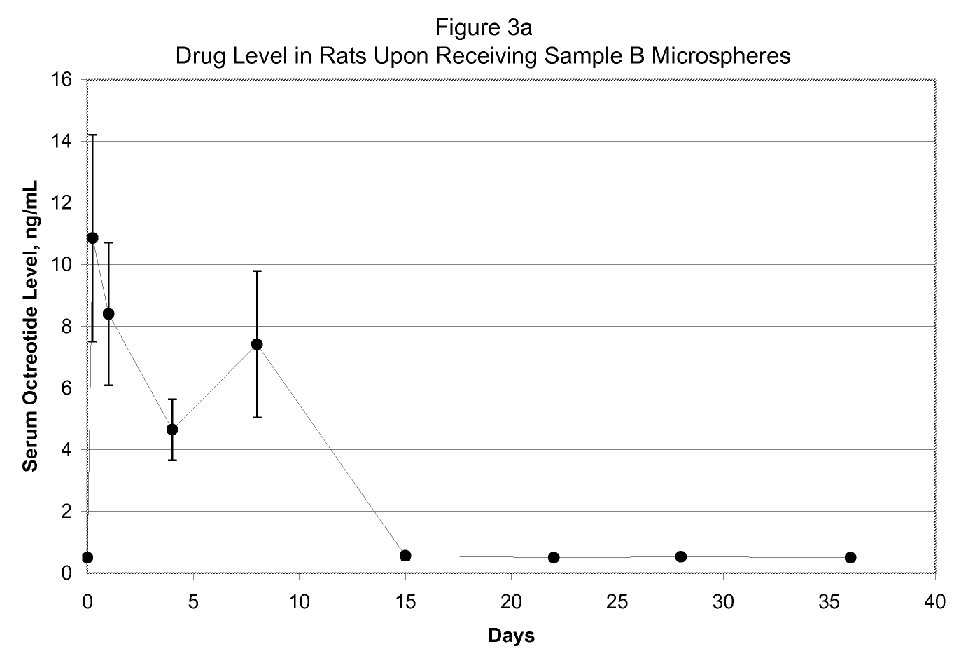

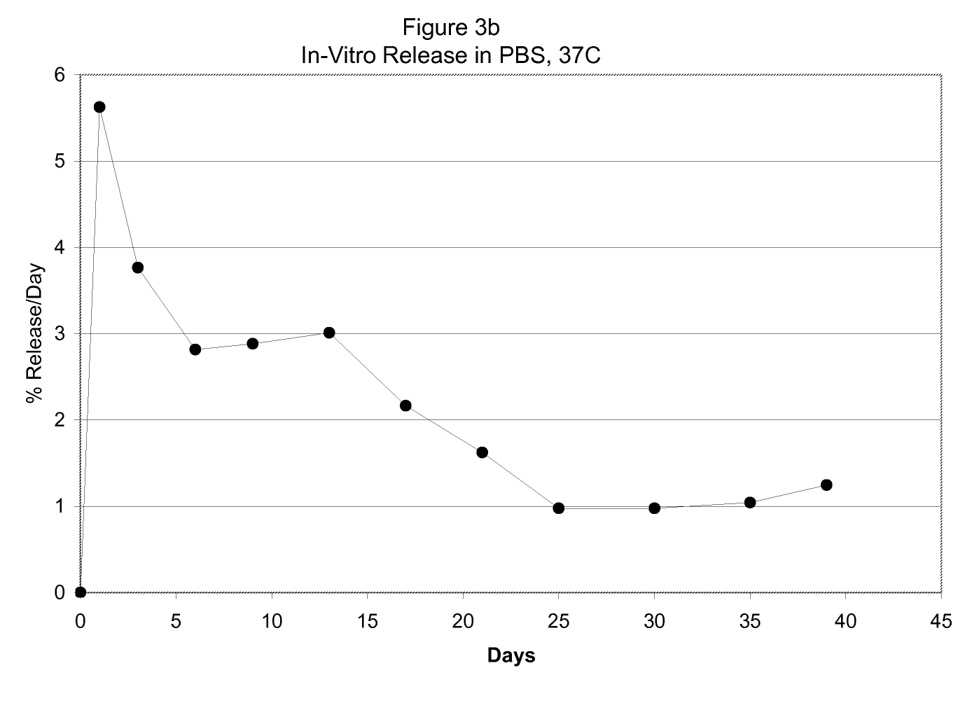

In another in-vivo study, rats were injected with Sample B, a RG502H polymer microsphere. Six rats received each microsphere formulation by subcutaneous (sc) injection and microspheres were suspended in diluents for injection. The dose was 1.5 mg octreotide acetate per rat. FIGS. 3b and 3a compare the in-vitro release from Sample B microspheres and the in-vivo release in rats, respectively. The results show that Sample B microspheres started releasing the drug immediately after injection and completed the release within 15 days.

50:50 DL2.5A Polymer Batch (Sample E)

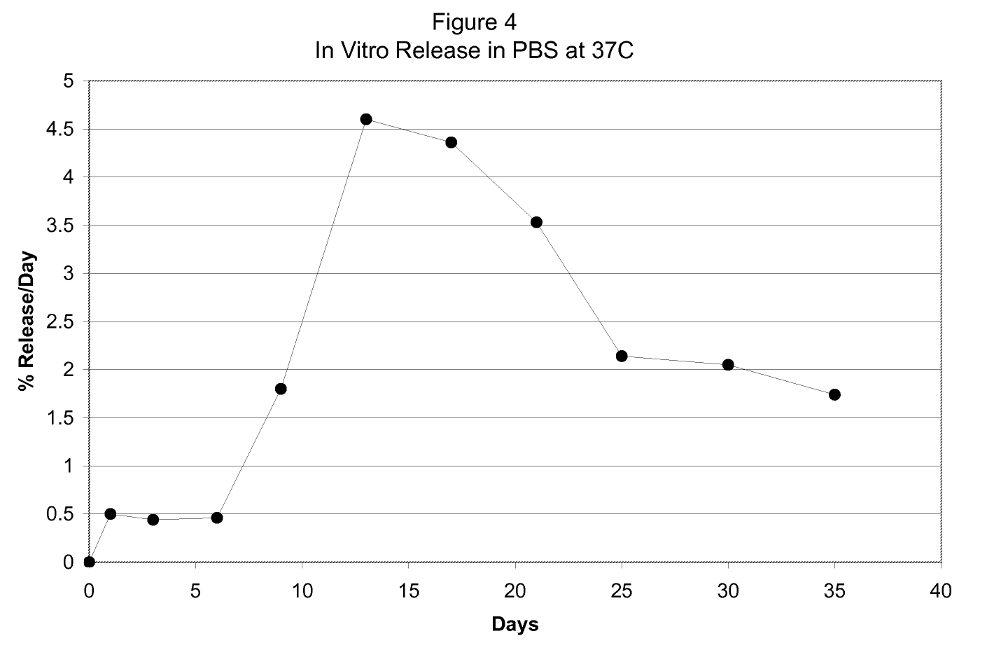

This batch was not subjected to an in-vivo release study. In-vitro release showed a small lag time for release (FIG. 4).

### EXAMPLE 2

In the next study, two microsphere batches prepared from PLGA 75:25 were evaluated for the drug release study. Table 3 compares the preparation parameters and properties.

TABLE 3
Microsphere Batches Prepared from PLGA75:25

| | | PLGA 75:25H | 75:25DL2.5A |
|---|---|---|---|
| | | G | H |
| DP Composition | Polymer | 0.24 | 0.20 |
| g/g | Octreotide | 0.03 | 0.03 |
| | DCM | 0.66 | 0.70 |
| | MeOH | 0.07 | 0.07 |
| DP Parameters | MeOH/DCM | 0.1 | 0.1 |
| | Target Load | 13 | 10 |
| CP Composition | PVA, g/g | 0.0035 | 0.0035 |
| Process parameters | Mixing speed | 7000 | 6500 |
| | Solvent removal | Air sweep, | Air sweep, |
| | | wash | wash |
| Finishing | Recovery of MS | Filtration | Filtration |
| Drug content in MS (%) | | 10.0 | 9.7 |
| Drug encap. Efiiciency | | 77 | 81 |
| Particle | 10% under | 2 | N.D. |
| Size, Volume | 50% under | 14 | N.D. |
| distribution, Micron | 90% under | 32 | N.D. |
| Bulk density | | 0.44 | 0.70 |
| % Impurity | | 2.9 | 8.0 |

“N.D.” is not determined

PLGA 75:25H Polymer, Batch (Sample G)

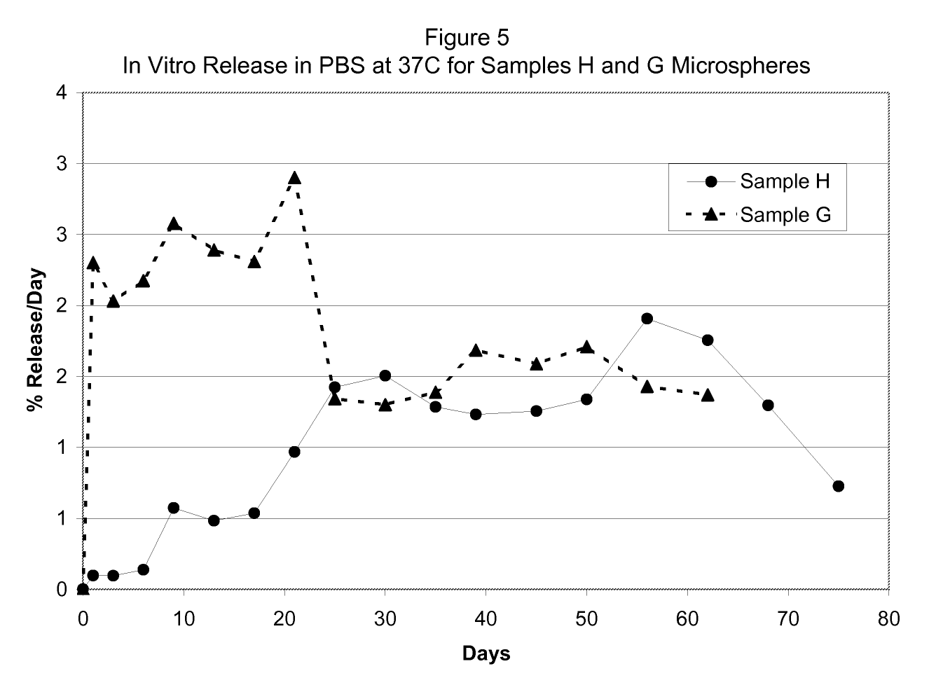

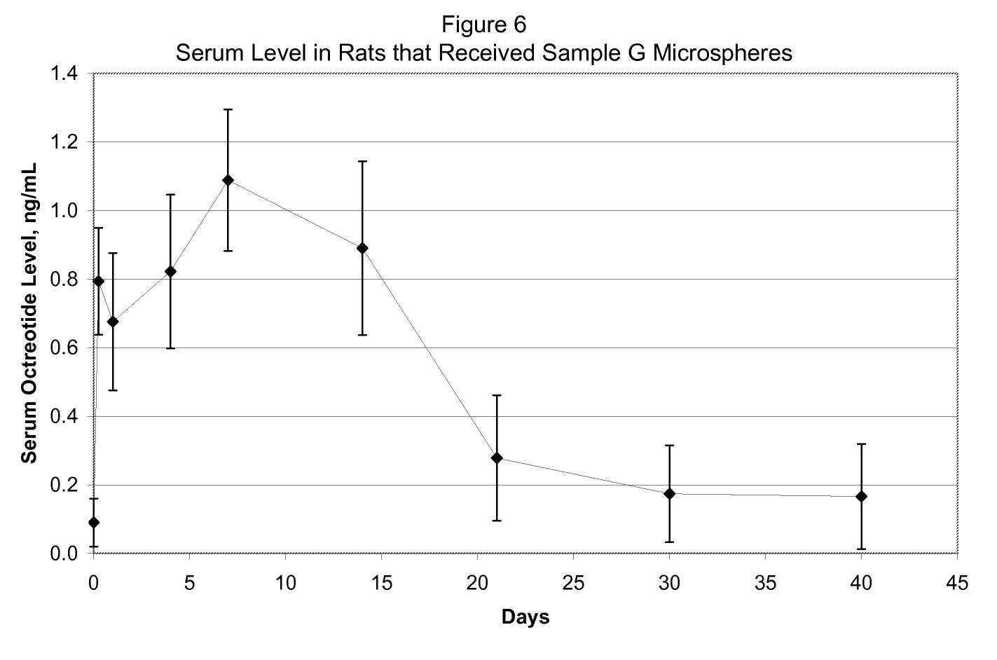

FIG. 5 shows in-vitro release for the microsphere batches using polymers of Samples G and H. FIG. 6 shows in-vivo release in rats using polymer of Sample G microspheres upon subcutaneous injection at the dose of 1.5 mg octreotide acetate per rat. The results show that PLGA 75:25, polymer batch of Sample G microspheres having a molecular weight of 14,000 released the drug soon after injection; FIG. 6 shows that release was completed in 20 days, which indicated this polymer was not ideal for a one month formulation.

PLGA 75:25 DL2.5A Polymer, Batch (Sample H)

PLGA 75:25 having a molecular weight of 25,000 had almost one week lag time for release; from this in vitro data the duration of the in vivo release was expected to be for about 45 days. Polymer in which 14,000<Mw<25,000 may be appropriate for starting the release with a minimum lag time and releasing the drug for a one month duration. The impurity level was higher for this polymer batch.

### EXAMPLE 3

In the next study, microsphere batch Sample I was prepared using PLGA 85:15 (molecular weight: 14,000). Microsphere batch Sample J was prepared from PLA polymer having a molecular weight of 7,000; microspheres Sample K was prepared from PLA polymer having a molecular weight of 14,000. Table 4 compares the preparation parameters and properties of the microsphere batches.

TABLE 4
Microsphere Batches Prepared from PLGA 85:15 and PLA

| | | | PLA | |
|---|---|---|---|---|
| | | 85:15DL2A | (Mw: 7 KDa) | PLA (14 KDa) |
| | | I | J | K |
| DP Composition | Polymer | 0.24 | 0.28 | 0.23 |
| (g/g) | Octreotide | 0.026 | 0.035 | 0.037 |
| | DCM | 0.66 | 0.61 | 0.62 |
| | MeOH | 0.08 | 0.07 | 0.11 |
| DP Parameters | MeOH/DCM | 0.12 | 0.12 | 0.18 |
| | Target Load | 10 | 11 | 14 |
| CP Composition | PVA | 0.0035 | 0.0035 | 0.0035 |
| (g/g) | | | | |
| Process parameters | Mixing speed | 6500 | 6000 | 6000 |
| | Solvent removal | Air sweep/wash | Air sweep/wash | Air sweep/wash |
| Finishing | Recovery of MS | Filtration | Filtration | Filtration |
| Drug content in MS (%) | | 9.1 | 9.3 | 10.3 |
| Drug encap. Efiiciency | | 91 | 85 | 74 |
| Particle | 10% under | 2 | 4 | 5 |
| Size, Volume | 25% under | 4 | 8 | 10 |
| distribution, Micron | 50% under | 11 | 14 | 19 |
| | 75% under | 19 | 20 | 26 |
| | 90% under | 24 | 25 | 31 |
| Bulk density | | 0.59 | 0.23 | 0.17 |
| % Impurity | | 0.1 | 0.23 | 0.54 |

85:15 DL2A Polymer, Batch (Sample I)

Microsphere batches prepared from PLGA 85:15 and PLA showed less than 1% impurities. Microsphere Sample I was not tested in rats; however in-vitro release showed that the formulation had appropriate initial release (no lag and no burst). Microsphere Sample I released the drug for approximately 60 days by in-vitro testing. Since in-vitro results are generally slower, in-vivo release duration is expected to be 45-50 days. One month release formulation is expected to be achieved with slightly lower molecular weight polymer.

Work with this 85:15 PLGA was extended further with additional batches and studies.

PLA Based Microspheres (Samples J and K)

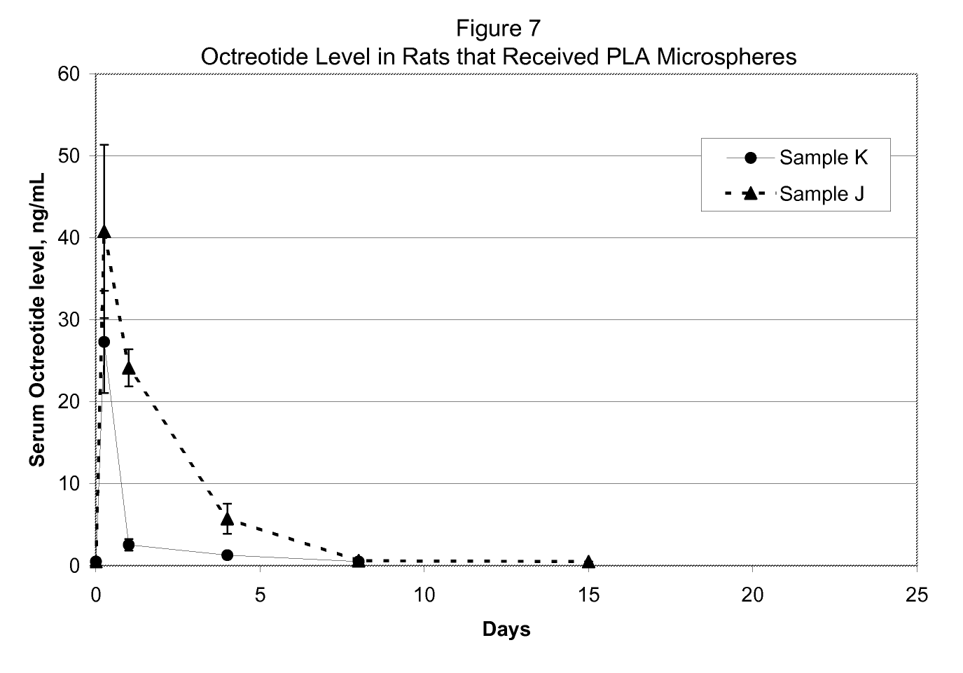

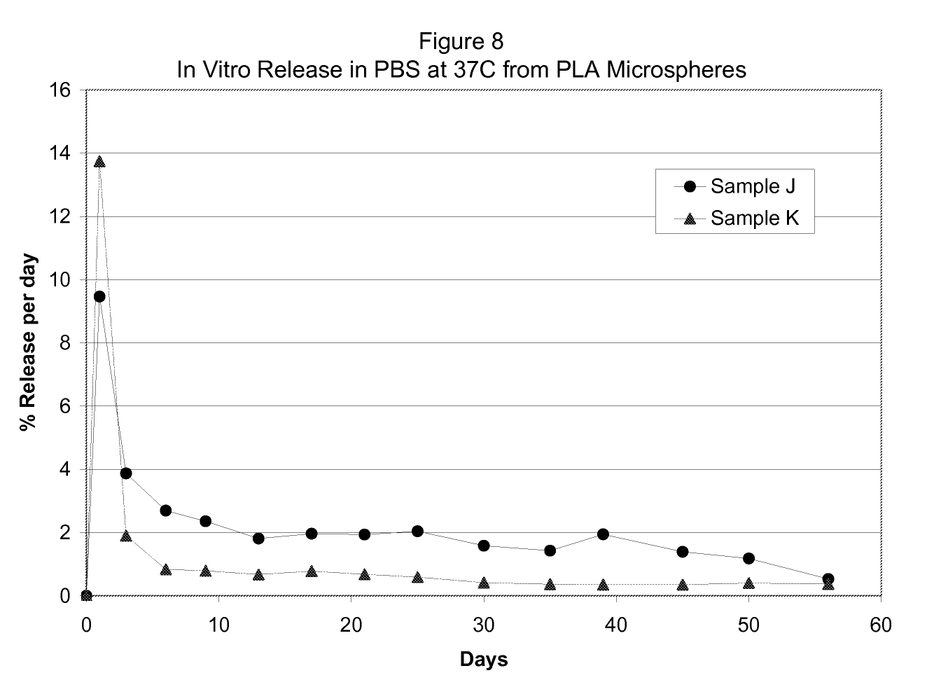

These batches were associated with a huge initial burst as shown by an in-vivo study upon injection at 1.5 mg octreotide acetate/rat (FIG. 7). An in-vitro release study also confirmed the higher initial burst and very slow release after the initial burst (FIG. 8).

Conclusions Regarding Polymers

To achieve drug release for one month duration the following polymers were studied: PLGA 50:50 having a molecular weight (Mw) between 30,000 and 45,000; PLGA 75:25 having a molecular weight of 14,000 and 25,000; and PLGA 85:15 having Mw under 17,000. As shown above, polymers having a higher glycolide content (PLGA 50:50) required higher molecular weight polymer for a one month release duration. However, it was found that higher molecular weight PLGA 50:50 polymer had a time lag for release. Hence, to achieve a formulation without a lag time for release PLGA 50:50 could not be used. Lower molecular weight polymer such as PLGA 50:50 having a molecular weight of 11,000 daltons did not have a lag time but released the drug within 15 days, which is unsuitable for a one month formulation.

PLGA 75:25 having a molecular weight of 14,000 daltons released the drug without a lag time, but completed release within 20 days. An in-vitro study showed that higher molecular weight polymer of PLGA 75:25 having a molecular weight of 25,000 daltons may release the drug for about 40 days duration. However, that was associated with an initial lag time for release, which in vitro data lead to the conclusion that the in vivo release period would be 50 days or longer.

PLGA 85:15 having a molecular weight under 17 kD released the drug for more than 30 days and was associated with a higher initial burst.

PLA was associated with a very high initial burst followed by a very slow release for a longer release duration. A one month release formulation with lower initial burst was difficult to achieve with PLA, even with low molecular weight PLA.

Sandostatin LAR uses the star type PLGA 50:50 in which the acid end groups are reacted with hydroxyl groups of the glucose unit. Free acid end groups are expected to be absent from the polymer. This is considered to be an acid end-blocked polymer. Blocking the acid end groups of PLGA could be performed with simple alcohols instead of glucose. Simple and mono functional alcohol produces acid end blocked PLGA/PLA retaining its linear characteristics. Such an acid end blocked polymer was used for comparative purposes in a formulation of this disclosure (O/W process). However, poor encapsulation efficiency was achieved (30% for RG503 compared to 90% for RG503H) and higher impurities (impurities were twice with RG503 polymer) associated with end blocked polymers. Therefore, free acid (unblocked) end group polymers are more suitable for octreotide microspheres according to the process herein.

### EXAMPLE 4

Octreotide Microspheres with 85:15 PLGA Polymer, Drug Load

To produce a formulation with appropriate initial release from PLGA 85:15, microsphere batches with varying target load (theoretical drug content) were investigated. This was achieved by varying the ratio of drug and polymer in the dispersed phase. Five batches with varying target drug loads (wt/wt) of 8.5, 10.0, 11.0 and 12.0% were prepared under similar preparation parameters. These batches were prepared using an in-line mixer for future scale-up and manufacturing.

Microsphere Preparation

The DP was prepared by combining the polymer solution in DCM and octreotide solution in methanol. The CP was prepared by dissolving PVA in water at elevated temperature (e.g., 70° C.). The CP was cooled to room temperature before microsphere preparation. Microspheres were prepared by delivering the CP at 2 L/min and the DP at 30 mL/min into the specially designed in-line Silverson mixer, as disclosed in the U.S. Pat. No. 5,945,126. The microsphere suspension was received in the solvent removal vessel (Applikon bioreactor). Washing and residual solvent removal was achieved by exchanging the CP with room temperature water, followed by hot water (30-40° C.), followed by room temperature water. The room temperature water removed external phase solvent; the hot water removed internal solvent and then the microspheres were returned to room temperature water. An air sweep was maintained to remove the headspace solvent during the solvent removal process. Washed and solvent free microspheres were collected by filtration on a membrane filter and dried at low temperature (>25° C.) under a vacuum.

Table 5 shows the preparation parameters of the microspheres, and properties of the microspheres. The drug incorporation efficiency for the microspheres was over 80% and the particle sizes were comparable. Bulk density was high enough for all 85:15 PLGA based microspheres; and the microspheres had suitable initial release. Low amounts of impurities were found. Total amount of impurities ranged from 0.6 to 1.7%.

TABLE 5
Preparation Parameter of the Octreotide Microsphere Batches

| Prep. Parameter | | L | M | N | O |
|---|---|---|---|---|---|
| Mw | | 9900 | 9900 | 9900 | 9900 |
| Batch Size | | 12 g | 12 g | 6 g | 6 g |
| DP | Polymer | 10.68 | 10.68 | 5.34 | 5.34 |
| Composition | Octreotide | 1.00 | 1.19 | 0.66 | 0.73 |
| (g) | DCM | 22.69 | 22.69 | 11.35 | 11.35 |
| | MeOH | 2.27 | 2.27 | 1.14 | 1.14 |
| DP | Target | 8.5 | 10.0 | 11.0 | 12 |
| | Load | | | | |
| Parameters | MeOH/ | 0.1 | 0.1 | 0.1 | 0.1 |
| | DCM | | | | |
| | Ratio | | | | |
| CP | PVA (g/g) | 0.0035 | 0.0035 | 0.0035 | 0.0035 |
| Composition | | | | | |
| Microsphere | CP/DCM | 140 | 130 | 130 | 130 |
| formation | Speed, | 6500 | 6500 | 7000 | 6500 |
| | RPM | | | | |
| Drug Load, % | | 7.4 | 9.4 | 10.2 | 9.8 |
| Incorp. Eff., % | | 87 | 94 | 93 | 82 |
| % Impurity* | | 1.66 | 1.22 | 0.57 | 0.65 |
| Particle Size, | 10% under | — | 3.59 | 2.1 | 2.1 |
| Volume | 25% under | — | 16.01 | 7.6 | 8.2 |
| distribution | 50% under | — | 24.02 | 19.8 | 21.9 |
| Micron | 90% under | — | 38.30 | 40.0 | 43.4 |

| | | | | |
|---|---|---|---|---|
| Bulk density, g/dL | 0.77 | 0.72 | 0.77 | 0.65 |

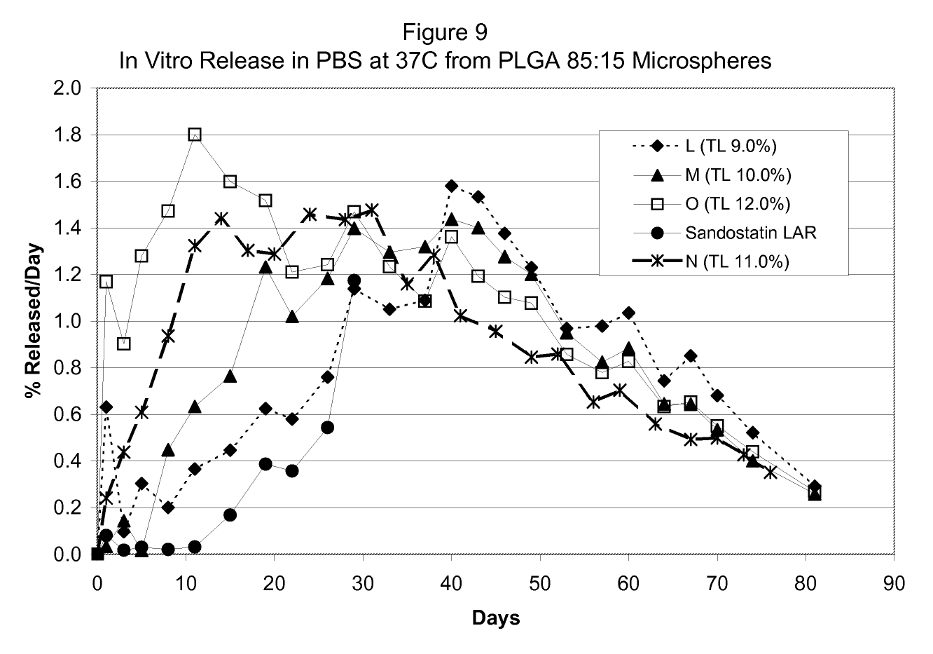

All the microsphere batches were subjected to in-vitro release testing. In vitro release of octreotide is shown in FIG. 9, plotted as percentage of octreotide released per day. In general, initial release increased as the drug load increased. After the initial phase (≈20 days), all microspheres released similarly.

### EXAMPLE 5

Octreotide-PLGA 85:15 Microspheres: Polymer Molecular Weight Variation and DP Variation

Using several PLGA 85:15 polymers with varying molecular weight, microsphere batches were prepared by O/W process using in-line mixer and washing was performed using hollow fiber filter by CP exchanges. A batch was also prepared with DMSO in the DP instead of methanol. All DP contained 5% or less glacial acetic acid for stability purposes.

TABLE 6
Microsphere Batches with Multiple Lots of 85:15 PLGA

| | | | | | DMSO in | High | |
|---|---|---|---|---|---|---|---|
| | Multiple polymer Lots | | | | DP | Mw | Low Mw |
| Polymer | P | Q | R | S | T | U | V |
| Mw | 11500 | 10300 | 11000 | 10500 | 11500 | 13900 | 7900 |
| MeOH/DCM | 0.10 | 0.10 | 0.10 | 0.10 | N/A | 0.10 | 0.10 |
| DMSO/DCM | N/A | N/A | N/A | N/A | 0.07 | N/A | N/A |
| % AA in DP | 4 | 4 | 4 | 4 | 5 | 4 | 4 |
| Target Load, % | 10 | 10 | 10 | 10 | 10 | 10 | 10 |
| Silverson | 5500 | 5500 | 5500 | 5500 | 6000 | 5500 | 5500 |
| RPM | | | | | | | |
| CP/DCM | 160 | 160 | 160 | 160 | 160 | 160 | 160 |
| Ratio | | | | | | | |
| Solvent | 1 Hr at | 1 Hr at | 1 Hr at | 1 Hr at | 1 Hr at | 1 Hr at | 1 Hr at |
| Removal | 35 C., CP | 35 C., CP | 35 C., CP | 35 C., CP | 35 C., CP | 35 C., CP | 35 C., CP |
| | Exchange | Exchange | Exchange | Exchange | Exchange | Exchange | Exchange |
| Actual Load, % | 7.7 | 7.8 | 7.7 | 7.6 | 9.4 | 8.1 | 7.9 |
| E. | 77 | 78 | 77 | 76 | 94 | 81 | 79 |
| Efficiency, % | | | | | | | |
| Bulk Density, | 0.70 | 0.61 | 0.74 | 0.65 | 0.56 | 0.70 | 0.41 |
| g/mL | | | | | | | |
| P. Size, | | | | | | | |
| micron | | | | | | | |
| 10% under | 2.76 | 3.16 | 2.80 | 2.59 | 4.4 | 2.19 | 5.4 |
| 25% under | 10.0 | 13.3 | 10.6 | 8.72 | 17.6 | 9.61 | 11.7 |
| 50% under | 23.1 | 25.4 | 24.9 | 21.6 | 34.0 | 20.8 | 22.1 |
| 75% under | 33.3 | 35.0 | 34.4 | 30.8 | 48.1 | 30.2 | 32.1 |
| 90% under | 41.5 | 43.6 | 42.1 | 38.3 | 61.3 | 37.8 | 41.4 |

As shown in Table 6, microsphere batches were prepared using polymers having molecular weight ranging from 7900 to 13900. Drug encapsulation efficiency and particle size remained similar for most of the batches.

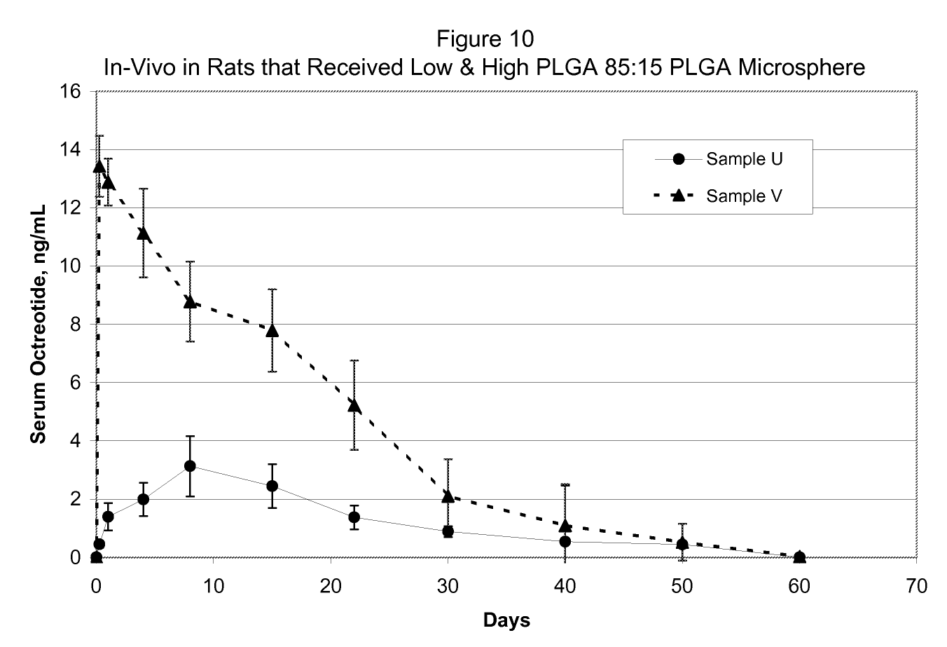

These microsphere batches were tested in-vivo in rats. FIG. 10 shows the results for the batches prepared with polymers having the highest and lowest molecular weight of and 7,900 daltons. Rats were dosed at 5 mg octreotide acetate per rat. The higher molecular weight polymer microspheres released for about 2 months and the lower molecular weight polymer microspheres released for about 20 days.

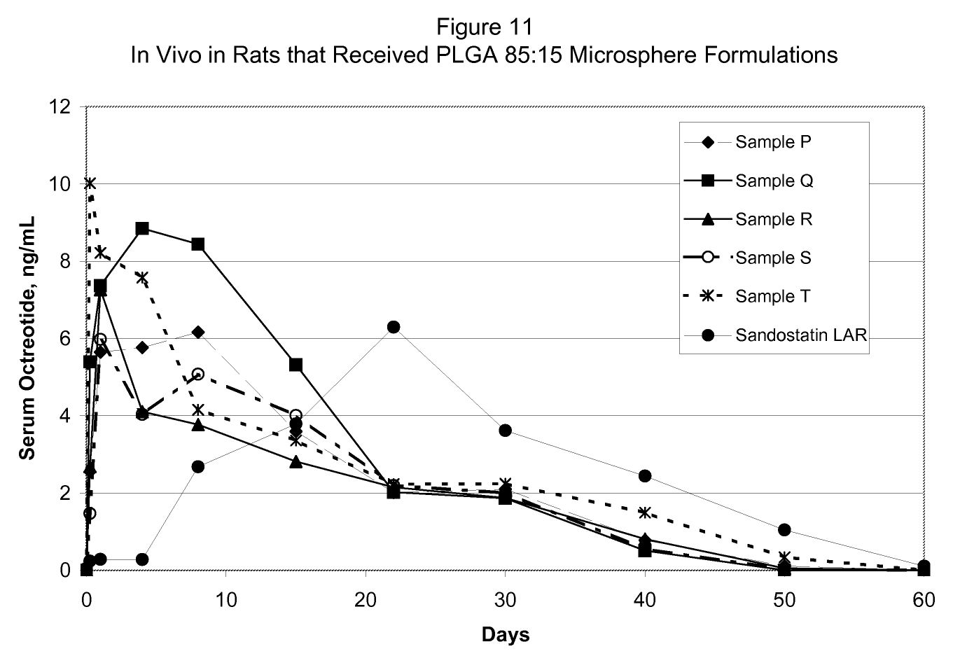

FIG. 11 shows the results for the batches prepared with multiple polymer lots and one sample with DMSO in the DP (Sample T). Sandostatin LAR was also tested in rats at the same dose and is provided for comparison. Rats were dosed at 5 mg octreotide acetate per rat. The results show that microsphere batches produced release without an initial time lag. Drug level in serum reached to >1 ng/mL in the first day for all the PLGA 85:15 PLGA batches and >3 ng/mL in the first day for all the one month release formulations. Sandostatin LAR, took days to achieve 1 ng/mL and >8 days to achieve >3 ng/mL. FIG. 11 shows that lower molecular weight polymer (Samples Q and S) could produce microspheres for 15-20 day release duration and higher molecular weight polymer (Sample P) could produce microspheres for nearly about two months release duration.

### EXAMPLE 6

Pilot Scale Dosage Form, In-Vivo Result

Microsphere Batch

A pilot scale batch was prepared using PLGA 85:15 polymer to achieve a dosage form in vials that also contain the diluent components, mannitol, carboxymethyl cellulose and polysorbate-80. A microsphere suspension in diluent was filled into vials to achieve 22 mg octreotide acetate per vial. Table 7 shows the preparation parameters and properties of the batch.

TABLE 7
Preparation Parameter and Properties of GC100903

| Parameter | Details | Value |
|---|---|---|
| Dispersed Phase | 85:15DL2AP (Mw: 11000) | 0.3379 |
| Composition (g/g) | DCM | 0.5287 |
| | MeOH | 0.0532 |
| | Acetic acid | 0.0407 |
| | Octreotide acetate | 0.0396 |
| CP Composition (g/g) | PVA concentration in CP | 0.0035 |
| Diluent composition | Carboxymethyl cellulose | 0.005 |
| (g/g) | (Na) | |
| | Mannitol | 0.0635 |
| | Tw-80 | 0.00076 |
| Microsphere | DP deliver rate | 22.1 g/min |
| Formation | CP delivery rate | 2000 g/min |
| | CP/DP Ratio | 90 |
| | Silverson mixing | 5500 rpm |
| Bulk microsphere | Actual Load (Bulk MS) | 7.8% |
| properties | Encapsulation Efficiency | 78% |
| | Bulk Density | 0.61 g/mL |
| Particle Size | 10% Under | 4.17 μm |
| | 25% Under | 10.3 μm |
| | 50% Under | 23.9 μm |
| | 75% Under | 37.8 μm |
| | 90% Under | 46.8 μm |
| Properties of | Finished Vial Dose | 23.38 ± 0.24 mg |
| finished vials | Free drug upon recon | 0.43% |
| | % Impurity | <0.5% |
| | % Moisture | 0.23% |
| | Syringeability | Syringeable through |
| | | 22G |

The microsphere batch was produced using the specially designed Silverson mixer as described earlier by flowing CP and DP under mixing at the rates shown in Table 7. The microsphere suspension was received in the solvent removal vessel. After the microsphere formation step, microspheres were washed with room temperature water followed by hot water (38° C.) and finally with room temperature water. Microspheres were suspended in diluent and the concentration of octreotide was determined by in-process assay. Based on the in-process assay value the suspension was filled into vials at 1.68 g/vial to achieve the target octreotide acetate concentration of 22.4 mg/vial. Vials were freeze dried under vacuum by freezing to −40° C. for 4 hours followed by a continuous ramp to +25° C. over approximately 33 hours. Terminal drying was performed at +25° C. for 10 hours.

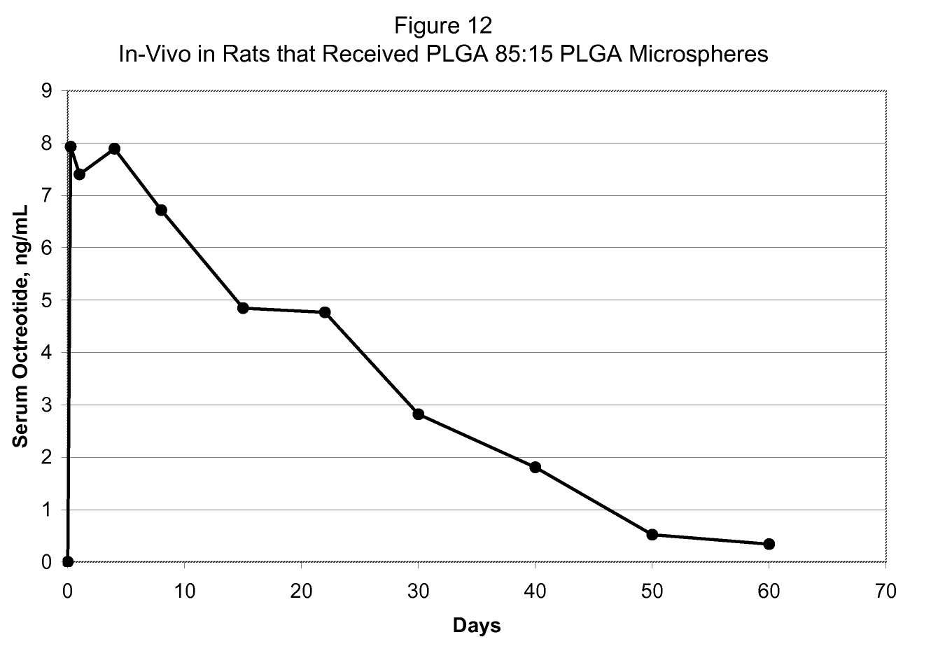

The product vial contained 23 mg octreotide acetate, 87 mg mannitol, 7 mg carboxymethylcellulose and 1 mg polysorbate-80. Freeze dried vials were reconstituted with 1.5 mL water forming a suspension that was syringeable (injectable) through a 22G needle. An in-vivo study injecting the microsphere suspension into rats at 5 mg dose per rat showed that the formulation released the drug for one month duration and released the drug without a lag time (FIG. 12). Microspheres released the drug to achieve >7 ng/mL within a day and maintained the level >2 ng/mL for a one month period. The vialed product was stable at room temperature for 24 months. The product also showed stability at 40° C. for six months and a small amount of impurity was produced. Drug release from the microspheres by an accelerated in-vitro test remained similar during the storage time indicating that the drug release performance of the product did not change.

Many modifications and variations of the invention will be apparent to those of ordinary skill in the art in light of the foregoing disclosure. Therefore, it is to be understood that, within the scope of the appended claims, the invention can be practiced otherwise than has been specifically shown and described.

What is claimed is:

1. Microspheres for releasing an octreotide compound without an initial time lag, comprising a poly(D,L-lactide-co-glycolide) polymer matrix in which a ratio of lactide to glycolide ranges from 80:20 to 90:10 mol %, said polymer having a molecular weight ranging from about 6000 to 16000, and wherein said octreotide compound is dispersed in said polymer matrix.

2. The microspheres of claim 1 wherein said polymer has an acid end group.

3. The microspheres of claim 1 wherein said ratio of lactide to glycolide in said polymer is 85:15.

4. The microspheres of claim 1 adapted for a 15 day formulation of said microspheres in which said polymer has a molecular weight of about 6000-8000.

5. The microspheres of claim 1 adapted for a one month formulation of said microspheres in which said polymer has a molecular weight of about 8000 to 14000.

6. The microspheres of claim 1 adapted for a two month formulation of said microspheres in which said polymer has a molecular weight of about 13000 to 16000.

7. The microspheres of claim 1 wherein said octreotide compound is octreotide acetate.

8. An injectable formulation comprising said microspheres of claim 1 and a diluent, said formulation being suitable for injection into a mammal in need thereof through a needle having an inner diameter of 394 microns or less.

9. The microspheres of claim 1 wherein said microspheres have an average size ranging from about 25 to 35 microns.

10. The microspheres of claim 3 wherein said microspheres are adapted to release octreotide acetate as said octreotide compound in serum of a rat to a concentration of >1 ng/mL in a first day of said release at a dose of 5 mg per rat.

11. The microspheres of claim 3 adapted for a one month formulation of said microspheres in which said polymer has a molecular weight of about 8000 to 14000, wherein said microspheres are adapted to release octreotide acetate as said octreotide compound in serum of a rat to a concentration of >3 ng/mL in a first day of said release at a dosage of 5 mg per rat.

12. The microspheres of claim 11 wherein said microspheres are adapted to release said octreotide acetate in serum of a rat to a concentration of >2 ng/mL at said dosage throughout a 30 day release period.

13. Microspheres for releasing octreotide acetate without an initial time lag, comprising a poly(D,L-lactide-co-glycolide) polymer matrix in which a ratio of lactide to glycolide is 85:15 mol %, said polymer having a molecular weight ranging from about 6000 to 16000 and an acid end group, and wherein said octreotide acetate is dispersed in said polymer matrix.

14. A method of administering an injectable octreotide compound to a mammal in need thereof without an initial time lag, comprising:
providing said microspheres of claim 1;
adding diluent to lyophilized said microspheres to form a first reconstituted formulation or providing said microspheres along with diluent in a lyophilized formulation;
reconstituting said lyophilized formulation with water to form a second reconstituted formulation; and
injecting said first or second reconstituted formulation to said mammal through a needle having an inner diameter of 394 microns or less.

15. The method of claim 14 wherein said diluent comprises mannitol, carboxymethyl cellulose and polysorbate.

16. A process for preparing microspheres for extended release of an octreotide compound without an initial time lag comprising:
providing poly(D,L-lactide-co-glycolide) in which a ratio of lactide to glycolide ranges from 80:20 to 90:10 mol %, said polymer having a molecular weight ranging from about 6000 to 16000;
a. preparing a dispersed phase by combining said polymer, dichloromethane, an octreotide compound, methanol, and acetic acid;
b. wherein a target loading of said octreotide compound in said dispersed phase ranges from 7 to 12% by weight;
c. dissolving polyvinyl alcohol in water to form a continuous phase;
d. mixing said dispersed phase in said continuous phase to form a microsphere suspension;
e. removing said dichloromethane, said acetic acid, said methanol and said polyvinyl alcohol from said microsphere suspension; and
f. removing residual dichloromethane and methanol from said microspheres by washing.

17. The method of claim 16 comprising exchanging diluent for said water, said diluent comprising sodium carboxymethylcellulose, mannitol and polysorbate to form an octreotide microsphere suspension.

18. The method of claim 17 comprising adjusting a concentration of said octreotide compound in said octreotide microsphere suspension.

19. The method of claim 18 comprising filling a formulation of said octreotide microsphere suspension into multiple containers and lyophilizing a single dosage of an entire said formulation in each of said containers.

20. The process according to claim 19 wherein said lyophilized octreotide formulation is a pharmaceutical formulation for injection.

21. The process of claim 16 wherein said target loading ranges from 9 to 11%.
